(19) United States
(12) Patent Application Publication
(10) Pub. No.: US 2017/0372441 A1
(43) Pub. Date: Dec. 28, 2017

(54) INFUSION PLANNING SYSTEM WITH CLINICAL DECISION SUPPORT

(71) Applicant: Smiths Medical ASD, Inc., Plymouth, MN (US)

(72) Inventors: Grant A. ADAMS, Anoka, MN (US); Eric WILKOWSKE, North Oaks, MN (US)

(73) Assignee: Smiths Medical ASD, Inc., Plymouth, MN (US)

(21) Appl. No.: 15/533,621

(22) PCT Filed: Dec. 3, 2015
(86) PCT No.: PCT/US2015/063710

§ 371 Date: Jun. 6, 2017

Related U.S. Application Data
(60) Provisional application No. 62/095,522, filed on Dec. 22, 2014.

(51) Int. Cl. G06Q 50/24; G06Q 10/06

(52) U.S. Cl. CPC G06Q 50/24; G06Q 10/06; A61M 2205/3561; A61M 2205/3576

(57) ABSTRACT

An infusion planning system providing clinical decision support for dynamic patient treatment scheduling. The infusion planning system includes a graphical user interface and a rescheduling assistance engine. The graphical user interface presents a time schedule display graphically representing a plurality of patient treatments over time including at least one infusion delivery profile graphic associated with an ordered infusion. The rescheduling assistance engine includes a user interface display providing a plurality of selectable schedule updates each comprising a set of recommended changes to the patient treatments in compliance with rules governing the patient treatment. The rescheduling assistance engine also provides a selectable graphical visual preview of each of the selectable schedule updates in some embodiments.

### TECHNICAL FIELD

Embodiments relate generally to improvements to systems and methods for coordination of medical care and planning, visualization, staffing, delivery, resource allocation, and documentation tools for medical care, including management of patient infusion pumps, in hospitals and other medical care facilities. More particularly, embodiments relate to systems and methods with enhancements to scheduling adaptability and efficient adjustments to deviations from scheduled patient treatments.

### BACKGROUND

Coordinating patient care accurately and efficiently within a hospital or medical facility can be complex and challenging. During the course of a day, a nurse, medical caregiver or clinician might be responsible for the care of multiple patients, each of whom could receive medication from multiple infusion pumps. Therefore, such medical personnel can be significantly burdened with keeping track of numerous historical, present, and planned future infusions. Further complicating the management of infusions is the fact that some such therepies need to be coordinated with other care and diagnostic activities, such as blood draws and lab work, MRIs, CAT scans, nutritional intake, and other infusion therapies, for example.

Systems have been proposed by the inventors of the present application to enhance efficiency and ease demands on medical personnel by providing these individuals with tools to better manage and understand the care they are providing to patients. Such systems may, for example, electronically import patient and treatment data and present timeline-based schedules and tracking of a patient or group of patients that is readily accessible and capable of visualization by the medical personnel. Associated rules regarding treatments, medications, and timing help to prevent errors and improve efficient use of resources. A disclosure of this type of proposed system can be found in PCT/US2014/044586 filed on Jun. 27, 2014 entitled “Infusion Planning System” which is hereby incorporated by reference.

Proposed and existing medical and infusion planning systems would benefit greatly from improvements that enable the systems to quickly reschedule and adapt medical treatments in response to, for example, rapidly changing patient conditions and available medical resources. Accordingly, improvements providing additional support to clinicians dealing with complex and readily changing medical environments are desired.

### SUMMARY

Embodiments relate to infusion planning systems that improve planning and visualization of patient care and include methods, systems, and apparatuses for planning, visualizing, and coordinating medication delivery. Specifically, embodiments provide systems, methods, and apparatuses with the ability to quickly and safely adapt to unanticipated or rapid changes in medical events, patient needs, and available resources. These embodiments provide scheduling capabilities for infusion pumps, medical devices, and other patient treatments allowing improved planning, recording, and reporting in hospitals or health care facilities.

One embodiment relates to an infusion planning system that provides adaptive clinical decision support for dynamic patient treatment scheduling. The infusion planning system includes a graphical user interface and a rescheduling assistance engine. The graphical user interface presents a time schedule display graphically representing a plurality of patient treatments over time including at least one infusion delivery profile graphic associated with an ordered infusion. The rescheduling assistance engine includes a user interface display providing a plurality of selectable schedule updates each comprising a set of recommended changes to the patient treatments in compliance with rules governing the patient treatments. Further, in certain embodiments the rescheduling assistance engine provides a selectable graphical visual preview of each of the selectable schedule updates.

Another embodiment relates to an infusion planning system that provides adaptive clinical decision support for dynamic patient treatment scheduling. The infusion planning system includes a computing platform interfaced with a computer network, the computing platform including computing hardware having a processor, data storage, and input/output facilities, and an operating system implemented on the computing hardware. The infusion planning system also includes instructions that, when executed on the computing platform, cause the computing platform to implement a graphical user interface and a rescheduling assistance engine. The graphical user interface presents a time schedule display graphically representing a plurality of patient treatments over time. The rescheduling assistance engine identifies and graphically presents recommended options for scheduling adjustments on the time schedule display of the graphical user interface that include at least one alternate sequence of revised patient treatment.

An embodiment is also directed to a further infusion planning system that provides adaptive clinical decision support for dynamic patient treatment scheduling including a graphical user interface for scheduling a plurality of patient treatments and a rescheduling assistance engine. The graphical user interface for scheduling a plurality of patient treatments includes scheduling a plurality of infusions delivered by at least one infusion pump. The graphical user interface includes a time schedule display comprising a plurality of columns representing time intervals during at least one particular date, a plurality of infusion bars each representing an ordered infusion for administration, and one or more infusion delivery profile graphics associated with each ordered infusion that depicts both an amount of medication to be delivered to a patient and a length of time period needed for delivery. Further, the infusion delivery profile graphics are configured for movement within the infusion bar associated with the corresponding ordered infusion, such that the infusion delivery profile graphics are aligned with the columns representing the time intervals over which the ordered infusion is scheduled for delivery. The rescheduling assistance engine provides a plurality of selectable schedule updates each comprising a set of recommended changes to the plurality of patient treatments in accordance with assigned rules governing each of the plurality of patient treatments. The graphical user interface provides visual previews of the selectable schedule updates.

Another embodiment is directed to a rescheduling assistance engine for use within a graphical user interface of a medical scheduling system. The rescheduling assistance engine includes a user interface display providing a plurality of selectable schedule updates each comprising a set of recommended changes to one or more patient treatments in compliance with rules governing the one or more patient treatments. Selectable graphical visual previews of each of the plurality of selectable schedule updates are provided and at least one of the one or more patient treatments is a medical infusion.

Embodiments are also directed to a method of updating a treatment schedule for a patient in an infusion planning system. The method includes constructing a visual schedule of anticipated patient medical treatments including one or more infusions in a graphical user interface of an infusion planning system. The method also includes receiving feedback data related to actual patient medical treatments performed on the patient. The method also includes detecting deviations from the anticipated patient medical treatments based on the feedback data received. The method further includes enabling a rescheduling assistance engine when deviations are identified. The method includes providing a plurality of options to modify the anticipated patient medical treatments. The method also includes rescheduling the anticipated patient medical treatments based on a selection of one of the plurality of options to modify the anticipated patient medical treatments provided by the rescheduling assistance engine.

An embodiment is directed to an infusion planning system for scheduling medical events including medical infusions. The infusion planning system includes a graphical user interface including a timeline-based graphical schedule of future patient treatments on a first portion of a displayed timeline. The graphical user interface also includes a timeline-based graphical schedule of past patient treatments on a second portion of the displayed timeline, configured to display verified past patient treatments and unverified past patient treatments, based on feedback received regarding a set of actual patient treatments administered. Some embodiments can include a real-time display of patient parameter feedback indicative of the current and past diagnostic states of a patient.

### BRIEF DESCRIPTION OF THE DRAWINGS

The disclosure may be more completely understood in consideration of the following detailed description of various embodiments of subject matter herein in connection with the accompanying drawings, in which:

FIG. 1 is an example of a medical environment depicting a need for scheduling and management of various infusion orders and medical events with an infusion planning system, according to an embodiment.

FIG. 2 is an example of a graphical user interface of an infusion planning system, according to an embodiment.

FIG. 3 is an example of a graphical user interface of an infusion planning system, according to an embodiment.

FIG. 4 is an example of a graphical user interface of an infusion planning system displaying past, present and future scheduling data, according to an embodiment.

FIG. 5 is an example of a graphical user interface including a pop-up display of a rescheduling assistance engine, according to an embodiment.

FIG. 6 is an example of a graphical user interface including a graphical visual preview provided by a rescheduling assistance engine, according to an embodiment.

FIG. 7 is a block diagram of an exemplary infusion planning system computing environment, according to an embodiment.

FIG. 8 is a flow diagram of an infusion planning method, according to an embodiment.

FIG. 9 is a flow diagram of a method of updating a treatment schedule for patients in an infusion planning system, according to an embodiment.

FIG. 10 is a flow diagram of a method of updating schedules in an infusion planning system, according to an embodiment.

FIG. 11 is a flow diagram of an infusion planning method, according to an embodiment.

### DETAILED DESCRIPTION

The various embodiments may be embodied in other specific forms without departing from the essential attributes thereof; therefore, the illustrated embodiments should be considered in all respects as illustrative and not restrictive.

Systems like the one disclosed by example herein, that are equipped with advanced scheduling and management of medical events combined with real-time or near real-time accounting of patient treatments, advantageously provide technological features that are important to the practice of medicine and patient care. Advanced scheduling systems generally provide the tools to effectively establish rules prior to medical treatments that govern those treatments. These rules can be used to avoid adverse events and alert medical practitioners to problematic combinations of medications/treatments and well as problematic timing of events related to those treatments. Moreover, advanced scheduling and management systems provide the potential benefit of optimizing the timing of treatments and maximizing hospital resources. These systems can lower the number of medications and treatments required by patients and provide better outcomes. For example, one type of medical treatment that is often scheduled in advance and could benefit from such improvements relates to infusions of medicaments and fluids to patients. Further, records generated by such treatments provide enormous potential to inform and improve future patient treatments and diagnosis.

Advanced scheduling and management systems, however, present their own challenges since they may be highly dependent on the occurrence and prompt reporting of time-based events to the system. Many medical treatments, by their nature, occur over time and frequently require unanticipated changes in patient condition, treatment, and hospital resources as well as rapid and timely responses to those unanticipated changes. In order for a real-time scheduling system to be effective, the system must quickly receive data regarding past treatment events and provide an effective way to quickly respond to variations in those events from a previous schedule. Moreover, safeguards involving a qualified medical professional must be incorporated to this system as a check on patient safety and to ensure the so-called “rights” of patient care are appropriately observed. Accordingly, a qualified medical professional must be readily equipped by the system with the information necessary and tools to make appropriate decisions promptly and with adequate authorization controls. Therefore, because a medical system operates in real-time, it is highly dependent on accurate and timely treatments as well as accurate and timely records of those treatments and variations and deviations therefrom. The novel and inventive subject matter of this disclosure recognizes these needs and challenges, and meets them by providing improved systems, tools and methods which enable timely responses to variations and deviations in scheduled medical treatment of patients.

Embodiments generally relate to a planning tool for medical infusions, patient care, and medical facility resources to improve efficiency and patient outcomes. Some embodiments will function as a day planner and scheduling tool for a medical caregiver such as nurse or other clinician, including a rescheduling assistance engine for efficiently addressing unanticipated events. In such embodiments, this day planner will allow the medical caregiver to efficiently and effectively coordinate hospital care, especially with respect to delivery of medicament and fluids via an infusion pump whether for one patient receiving multiple treatments or multiple patients receiving single or multiple treatments, even when last minute changes in treatment and schedule occur.

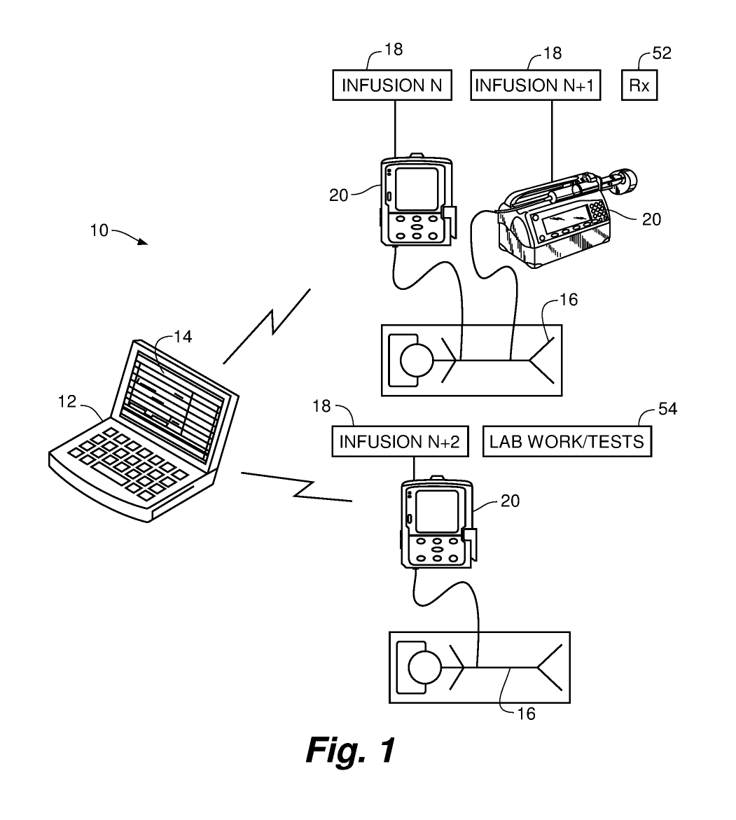

There are numerous environments and situations in which an infusion planner would be useful to a nurse or other medical caregiver or professional. FIG. 1 shows a representative example of a patient care arrangement in a hospital or medical facility that could require different types of planning and capabilities to appropriately coordinate care for ordered infusions or medical events. This arrangement generally depicts infusions or medical events that require time periods to complete that are typically prescribed or known in advance.

In particular, FIG. 1 depicts an example of a medical environment 10 in which a medical caregiver device 12 displaying a graphical user interface (GUI) 14 is shown. The device 12 and GUI 14 could be part of a larger scheduling/planning system that visually displays the timing of fluid delivery to one or more patients 16. Specifically, here the scheduling/planning system presents a display on which a medical caregiver can simultaneously visualize fluid delivery for multiple patients 16, each having one or more infusions 18 administered. The types of fluid delivery represented here include multiple infusions 18 from one or more infusion pumps 20 or types of infusion pumps 20. The medical caregiver device 12 can be a PC workstation of a nurse or clinician in a hospital. Alternatively, the medical caregiver device 12 can be a laptop, electronic tablet, smart phone, custom controller or other display and processing device providing a GUI 14. The infusion pumps 20 can be the same type or different types of pumps. Further, some types of infusion pumps 20 can be capable of delivering multiple infusions 18. The medical caregiver device 12 can operate autonomously from the pumps 20 in some embodiments, can be directly communicatively connected by wired or wireless connection to the pumps 20 themselves in some embodiments, or be communicatively coupled to a server or network in communication with the pumps 20 in other embodiments.

FIG. 1 also takes into account additional medication or medical events not necessarily related to medical infusion therapies. Specifically, taking non-infusion medications 52 such as pills or oral medication, or lab work and test procedures 54 such as blood draws, lab work, MRIs, and CAT scans, can be implemented as part of the scheduling/planning system and GUI 14 of the caregiver device 12. As the timing of these types of medical events will restrict the scheduling of or make administration of infusion therapies at particular times more or less desirable, it is beneficial to visually account for such events when planning a particular schedule for a patient. For purposes of this disclosure, the terms “patient treatments” or “medical treatments” referenced can broadly encompass any medical events, medical infusions, and other patient related activities that are scheduled for or recorded and related to patient care.

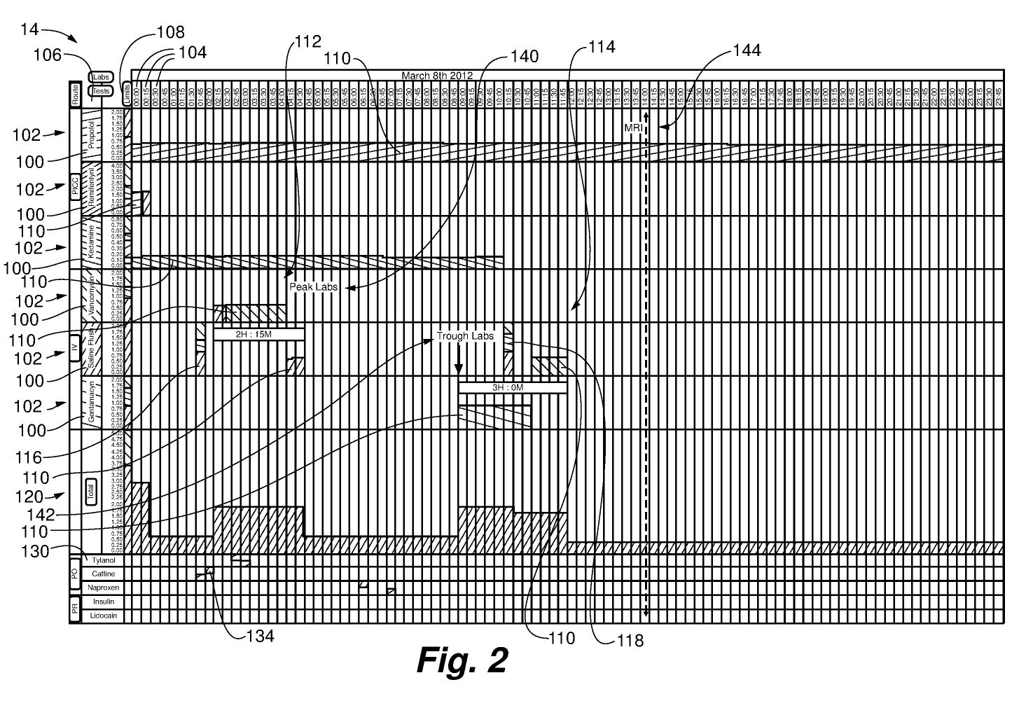

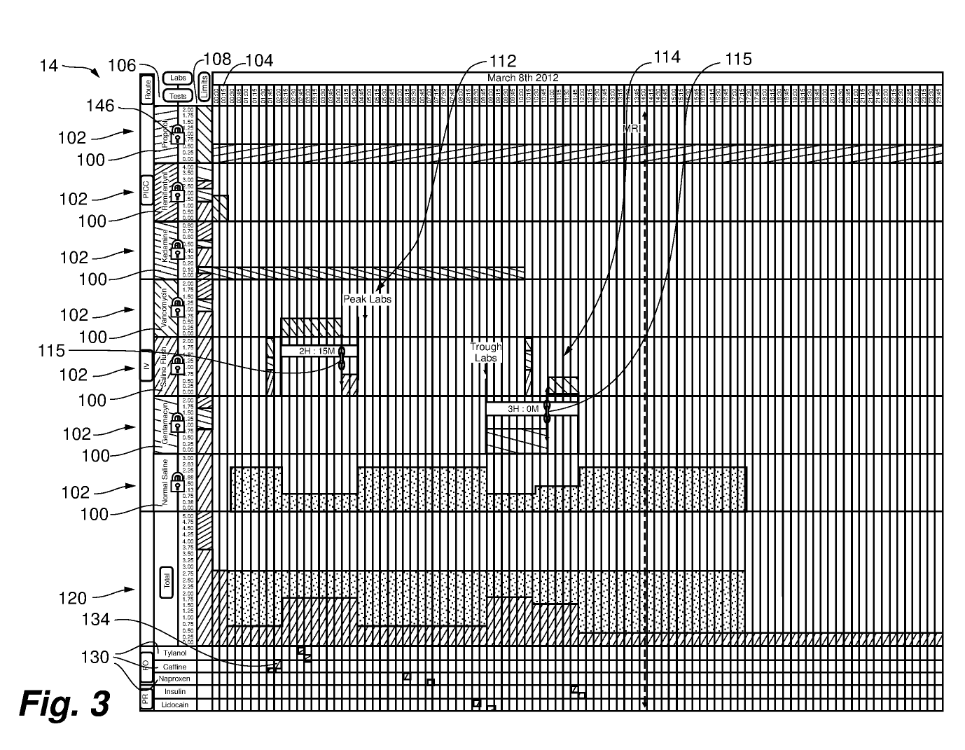

FIGS. 2 and 3 each show an example of an embodiment of a GUI 14 comprising a display for an infusion planning system providing a visual interface in which multiple infusions and medical events are depicted as a function of time. Ordered infusion therapies 100 for delivery are represented by a plurality of horizontally disposed rows providing horizontal infusion bars 102 set against a timeline composed of vertical time columns 104 each representing a segment of time for a particular time of day on a particular date. In FIGS. 2 and 3, each of these vertical time columns 104 are shown to represent a fifteen minute period on Mar. 8, 2012, for example. On the left side of the GUI 14, a column 106 which names the various infusion therapies is present in its own color coded segment. Accordingly, each respective infusion therapy 100 is listed within the corresponding horizontally disposed bar 102 for that infusion therapy 100. For example, in FIG. 2 the named infusions 100 shown are Propofol, Remifentynl, Ketamine, Vancomycin, Saline Flush, and Gentamicin. Adjacent the column 106 of named infusions 100 is a color-coded scale 108 reflecting the acceptable range of fluid delivery amount for the various infusions. These ranges provide the medication safety limits for the respective infusion. To the right of each of the listed infusions 100 and color-coded scales 108 are infusion delivery profile graphics 110 for the various infusions 100. The infusion delivery profile graphics 110 comprise or include a series of generally adjacent bars of varying heights. The combination of these bars as an infusion delivery profile graphic 110 provides both time of fluid delivery information based upon the width of the combined bars and volume of fluid delivery information based on their height. These graphical representations help a nurse or medical caregiver to better visualize and plan for patient care, particularly when known or restricted amounts of time are required for infusions.

It is contemplated that in some embodiments the infusion bars 102 can be disposed in a direction that is non-horizontal as depicted in FIGS. 2 and 3. The axis scale representative of infusion amount is shown along a vertical first ordinate in FIGS. 2 and 3, although other orientations for this ordinate are possible as well. The axis scale representative of time is shown along a horizontal second ordinate in FIGS. 2 and 3, although other orientations for this ordinate are contemplated as well. The vertical disposition of the columns 104 in the figures depicting various time intervals should not be viewed as limiting the orientation or shape of such features. Moreover, GUIs having depictions of various orientations and shapes are contemplated by this disclosure.

At times, one or more infusions are linked with one another, such as in the case of an antibiotic like Vancomycin or Gentamicin which may require subsequent infusion of a saline flush after its infusion. Accordingly, these two infusions are linked together as the saline flush is used to push any remaining medication to the patient 16. These two linked infusion examples 112 and 114 are respectively depicted in both FIGS. 2 and 3. FIG. 3 further includes a chain-link graphic 115 in GUI 14, as one optional way to further visually denote the linked relationship of infusions. This linking not only allows the scheduling of these infusions to be better understood and coordinated, but the linking also allows the medication safety limits for the earlier infusion (such as Vancomycin or Gentamicin in this example) to be applied to the subsequent saline flush. This can also be understood in a visual way as additional color coded limit bars 116 and 118 are respectively depicted within the saline flush infusion bar 102 timeline. Further, the visual representation of the antibiotic with saline flush is shown together as a single therapeutic event as a function of time. A horizontal bar depicting the cumulative total of infused medication and fluid is set forth at total infusion bar 120.

While the timing of some infusions is scheduled to be fixed in time, other infusions are set up such that they are allowed to “float” or be controllably variable. This is useful in a situation in which a patient, such as a neonate, has a fixed maximum volume of fluid that can be delivered at a time. A depiction of this can be understood from the GUI 14 in FIG. 3. Here, the total value for volume of fluid in the total infusion bar 120 is fixed; however, the volume of Normal Saline at bar 122 is allowed to float such that the combined total of the infusion remains constant.

In FIGS. 2 and 3, medications are shown on the GUI 14 which are administered by non-infusion methods as well. This can include pills or oral medication for example. Horizontal bars 130 for these types of medication are provided at the bottom portion of the chart in FIGS. 2 and 3. Administration of these substances is not depicted with a graphical representation of their dosage or volume, as in the infusion profiles above, but simply with a colored marker 134 at the time of their administration. For example, orally administered Tylenol, Caffeine and Naproxen are represented by indications, like markers 134, at appropriate times within bars 130.

Additional useful, time-based events can be placed on the medication timeline as well. These can include various types of medical care events unrelated to infusions such as, for example, blood draws, lab work, MRIs, and CAT scans. As referenced above, these medical care events, as well as ordered infusions and others, may be broadly referred to by the terms “patient treatments” or “medical treatments” for purposes of this disclosure. In the examples shown in FIGS. 2 and 3, an arrow and label for a peak lab draw is shown at 140, an arrow and label for a trough lab draw is shown at 142, and an arrow and label for an MRI is shown at 144. The arrows or graphics depicted can extend across all other infusions, as in the case of the MRI indications 144, or can alternatively only be shown proximate those infusions 100 potentially related to that event, as with the peak and trough lab draws 140 and 142. These events are visually depicted so that staff can coordinate the full care of the patient timely and efficiently, especially with respect to infusions. For example, peak and trough measurements need to take a certain time with respect to the administration of the medication to help ensure the validity of the data obtained from the test. Likewise, knowing that a patient must be moved to perform an MRI allows a caregiver to schedule infusions such that the minimum number of infusions are running during the transport process.

In some embodiments, certain users can have the ability to lock the scheduled delivery of particular infusions 100 on one or more horizontal infusion bars 102 of the GUI 14. Infusions 100 that are locked can contain a visual graphic such as a padlock 146 adjacent the column 106 of corresponding named infusion 100. Accordingly, when an infusion 100 is locked, no graphical changes can be made to that horizontal infusion bar 102 until the infusion 100 is unlocked. This lock feature enables a user to more easily set and understand which infusions must or should occur at certain times so that only the remaining combinations of scheduled infusions can be changed. This allows more easily and effectively scheduled infusions and helps to prevent mistakes in rescheduling and planning of infusions at unwanted or unworkable times.

The GUIs 14 shown in FIGS. 2 and 3 are merely examples of embodiments of possible configurations and appearances for the type of scheduling device contemplated. Other arrangements for differently displaying infusions and medical events are contemplated as well. Other embodiments can display multiple patients receiving infusions in similar embodiments. GUI displays 14 that include a large plurality of patients and/or infusions can require a display that reduces the amount of infusion data viewable or is otherwise downwardly-scalable to accommodate the number of patients or infusions shown.

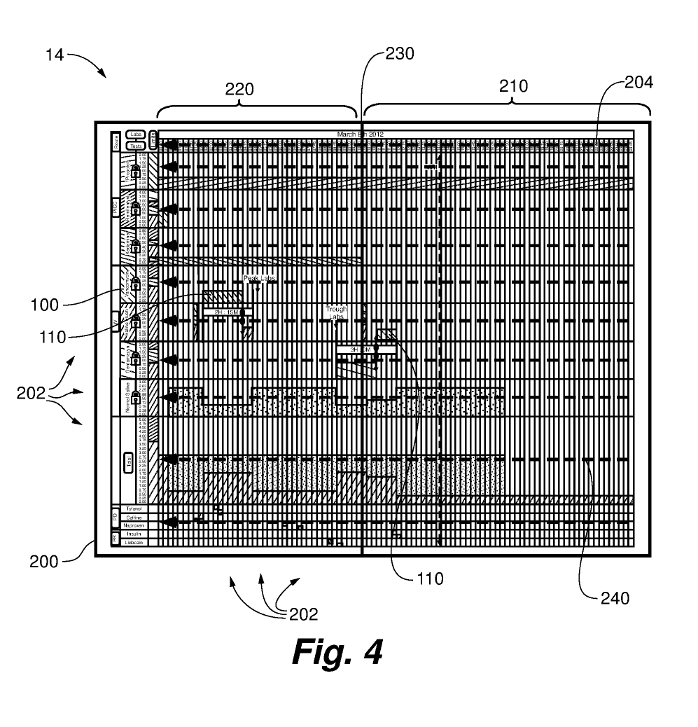

FIG. 4 depicts an example of a GUI 14 having a time schedule display 200 with both forward-looking planning and backward-looking recording and reporting of patient treatments 202 including infusions and medical care events. The time schedule display 200 can be real-time or near real-time in some embodiments. In the embodiment shown in FIG. 4, the display 200 is accomplished with a split or tiled screen having a timeline 204 extending horizontally across the screen. The display 200 includes a timeline-based graphical schedule of future patient treatments 210 on the right hand portion of the display and a timeline-based graphical schedule of past patient treatments 220 on the left hand portion of the display. Further, a central line 230 separates the schedules of future and past patient treatments 210 and 220. The central line 230 corresponds to the present time on the timeline 204 at the top of the display. In such a display, the timeline 204 and patient treatments including the infusion delivery profile graphics 110 associated with a particular ordered infusion 100 and other events will generally move horizontally from right to left across the display screen as time passes. Accordingly, the central line 230 will generally remain stationary during these movements. Embodiments will generally include at least one infusion delivery profile graphic 110. In FIG. 4, a number of dashed arrows 240 are shown to indicate this movement, although the arrows 240 here are only intended for illustration and are not part of the actual display. Accordingly, future planned infusions and medical events as well as recently-recorded actual infusions and medical events can be concurrently displayed on the same screen or GUI.

The central line 230 at the vertical split between these two displays represents the present time that a user is viewing the display 200. The line 230 representing this split is shown as a wide and/or dark line in FIG. 4, however, the width and other visual attributes of this line is preferably negligible or even non-existent or suppressed in some embodiments.

The right portion of the screen depicts a schedule 210 of times for planned infusions and other patient treatments 202 in the future. Future planned schedules of patient treatments 202, including infusions, are set by a nurse or medical caregiver and can be readily manipulated comfortably before their occurrences to enhance the efficiency and efficacy of patient treatment. The graphical representations help a nurse or medical caregiver to better visualize and plan for patient care. Rules governing drug interactions and timing protocols are built into the future schedule and restrict the types, way, amount, and timing of future medical treatments 202.

The left portion of the display depicts a schedule 220 of actual, delivered patient treatments 202 including past infusions. Feedback provided to the system can dictate the graphical depictions appearing on the actual schedule 220. Some of this feedback is sent into the system electronically by infusion pumps 20 or other devices or systems that automatically report their patient treatment activities. Other feedback is not automatic or nearly instantaneous, however. Some feedback, for example, may require manual input by a medical caregiver to enter or verify delivery of a particular patient treatment 202. Accordingly, such additional non-real time feedback may be necessary to effectively chart and record a patient treatment 202 within the infusion planning system. Although not specifically shown in FIG. 4, some graphical user interfaces can additionally display real-time patient parameter feedback that highlights the current and past diagnostic state of a patient. Accordingly, a real-time display of patient parameter feedback indicative of the current and past diagnostic states of a patient can be displayed and utilized by a user of the infusion planning system. In some embodiments, the graphical user interface can include a display having historical and real-time parameter feedback of a patient indicative of responses to one or more clinical changes.

Non-real time reporting can utilize a number of features to help visually distinguish between events. In some embodiments, the left side portion of the display, comprising schedule 220, may temporarily depict anticipated past treatments before they are able to be verified. Prior to receiving feedback to supply provide verification, the anticipated past treatments may be distinguished from those that are actually verified by system feedback. For example, unverified anticipated past treatments may be shown in grayscale and the verified patient treatments may be shown in color. Alternatively, unverified past anticipated treatments may be shown in a flashing state to the user, while verified treatments may appear as solid graphics. When an authorized medical caregiver is able to verify a treatment, he or she can convert the appearance from grayscale to color or from flashing to non-flashing, for example.

Several challenging factors, therefore, have a potentially significant impact on operation of the infusion planning system. First, the ability to effectively utilize the infusion planning system relies heavily on the ability to input prompt and timely feedback into the system. If a user cannot observe the events that have occurred without significant time delay, the appropriate and effective future schedule of treatments for a patient might remain largely unknown. The infusion planning system's effectiveness and efficiency can, accordingly, be closely aligned with the system's ability to receive timely and accurate data regarding treatments as well as patient data. Accordingly, embodiments of the present disclosure provide a system that has an enhanced ability to receive timely and accurate data.

A second challenging factor relates to the speed at which changes must be made in a medical treatment setting and the frequency of those changes. Deviations from expected patient treatment can occur rapidly in response to, for example, patient data indicating adverse effects to treatment or ineffective treatment. For example, a patient's blood pressure or heart rate may suddenly fall, or spike or rise; or another patient parameter may cause alarms or concerns to medical caregivers. Changes and adaptations to improve patient response can be constantly evaluated and modified. Further, deviations may occur due to malfunctions of medical equipment, untimely acting physicians or medical workers, and labs or testing equipment which are delayed or off schedule, etc. Accordingly, an inability to readily implement these deviations into the infusion planning system schedule can, in some instances, be limiting factors as well. One complicating aspect of this rescheduling for deviations is the dependent nature of one treatment in relation to one another. Similarly, potentially intertwined relationships between limited hospital resources and potentially numerous patients requiring those resources at similar times must be taken into account. Due to the interrelated nature of these needs, a change to one scheduled treatment for a patient may have cascading implications to the schedule of other treatments for that patient as well as other patients. Accordingly, various embodiments of the present disclosure recognize, address, and overcome these challenges.

Specifically, embodiments provide an easy to use system that equips a medical caregiver with the ability to rapidly recognize unverified medical events and to easily verify these medical events for the system so that accurate, up-to-date records and information are available for planning Embodiments provide an efficient system for rescheduling patient treatments and events when errors or deviations occur that require rapid changes to patient treatment scheduling. The system is designed to provide an accounting and visualization of cascading changes across multiple treatments and hospital resources for one or more patients.

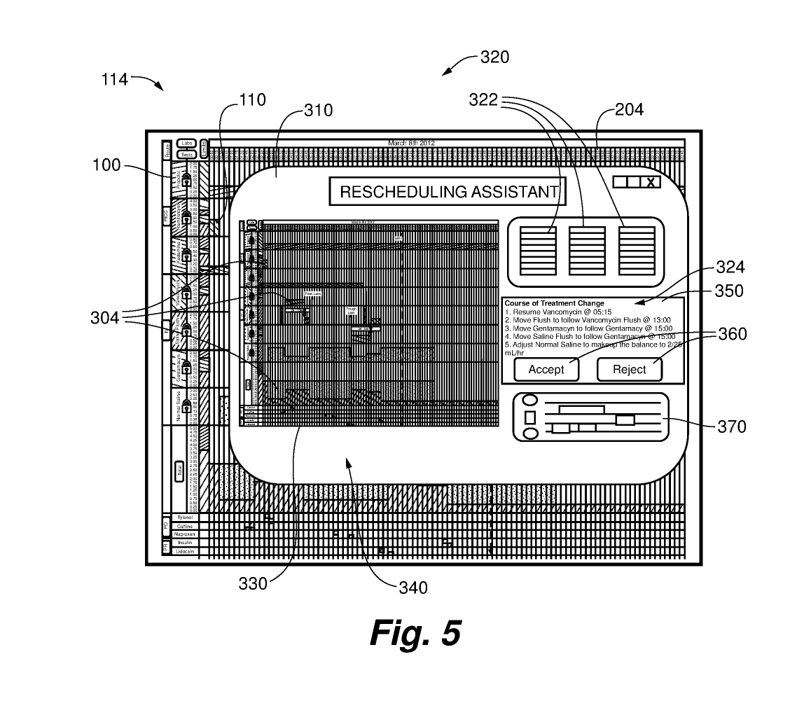

FIG. 5 shows an example of a GUI 14 as well as a user interface display 310 of a rescheduling assistance engine 320. In various embodiments, the infusion planning system includes a GUI 14 and a rescheduling assistance engine 320. As discussed above, the GUI 14 presents a time schedule display that graphically represents multiple patient treatments 304 over time, including infusion delivery profile graphics 110 associated with ordered infusions 100. In general, the user interface display 310 of the rescheduling assistance engine 320 provides a plurality of selectable schedule updates 322 for a patient schedule. Selectable schedule updates may also be referred to as options for scheduling adjustments in this disclosure as well. These selectable schedule updates 322 may take on various forms, graphics, descriptions, icons, table entries, etc. Each of the selectable schedule updates 322 is associated with and is made up of a set of recommended changes 324 to the patient treatments 304 in accordance with the rules governing the patient treatments 304; and such changes 324 may also advantageously be presented within to ensure the aforementioned “rights” of patient care. Each set of recommended changes 324 may be separately presented in a grouping or listing of a course of proposed treatment changes 350. The rescheduling assistance engine 320 can include a selectable graphical visual preview 330 of each of the selectable schedule updates 322 as well. The selectable graphical visual preview 330 in some embodiments can comprise a separate visual schedule depiction 340, as shown in the user interface display 310 comprising a pop-up type window illustrated in FIG. 5. Alternatively the selectable graphical visual preview 330 may be overlaid on or incorporated into the existing time schedule display 200 of the GUI 14 display itself, and does not require such a pop-up window or entirely separate scheduling window.

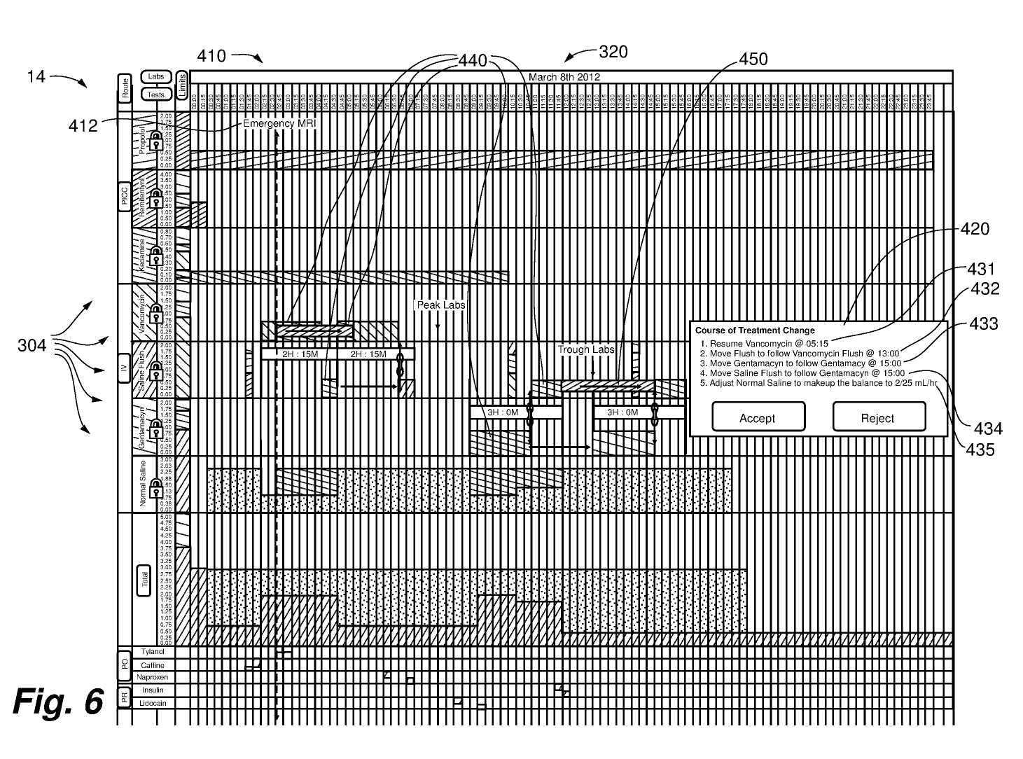

FIG. 6 shows an example of a user interface display 410 incorporated into a GUI of an infusion planning system. The user interface display 410 includes and is utilizing a rescheduling assistance engine 320. The user interface display 410 shown could operate on the GUI 14 for the infusion planning system to provide previews of scheduling changes. Alternatively, a user interface display 310 could encompass a pop-up display including a graphical visual preview 330, similar to the one shown in FIG. 5, for example. The user interface display 410 discussed in FIG. 6 will be referenced as an interconnected display system with GUI 14 for simplicity, but such a display is not limited to this type of arrangement. Accordingly, FIG. 6 shows one way in which possible scheduling changes of a rescheduling assistance engine 320 can be graphically depicted.

Specifically, FIG. 6 shows a scenario in which a deviation from the previously planned schedule occurred due to the scheduling of an unanticipated or emergency MRI. Namely, the GUI reflects that at 2:45 AM a patient receiving infusions of both Vancomycin and Normal Saline, had those infusions interrupted to conduct the emergency MRI. Accordingly, the GUI 14 displayed represents a rescheduling assistance engine 320 at work at or about 2:45 AM when the emergency MRI has just been scheduled.

In this example, the infusion planning system recognizes the need for rescheduling assistance, based on the insertion of an Emergency MRI event 412 that is in conflict with other planned infusion events. For purposes of this example, it can be assumed that the need for this MRI is made based on a clinical decision made outside of the schedule. However, in some embodiments feedback data and decision support could be used to fully advise and control the system to trigger an event or change in infusion protocol. For example: a real time blood sugar level could be used to trigger dosing of insulin; blood pressure, heart rate and total volume monitoring could trigger a regiment of inotropes, vasopressors, vasodilators, and diuretics to help manage the patient's cardiac and fluid states; real time pain monitoring could be used as a closed loop input for opioids; and patient feedback could trigger the need for an emergency procedure that shifts medication administration.

In the example of FIG. 6, the need for rescheduling could be realized based on a plurality of different types of feedback to the system. Once this deviation from planned patient treatment or other need is recognized, the rescheduling assistance engine identifies and graphically presents recommended options for scheduling adjustments on the time schedule display of the graphical user interface 14. At least one alternate sequence of revised patient treatments 304 is graphically provided by the rescheduling assistance engine.

For purposes of this disclosure, the term “engine” can be defined as a real-world device, component, or arrangement of components implemented using hardware, or as a combination of hardware and software, such as by a microprocessor system and a set of particular program instructions that adapt or prompt the engine to implement the particular functionality, which (while being executed) transform the microprocessor system into a special-purpose device. A engine can also be implemented as a combination of the two, with certain functions facilitated by hardware alone, and other functions facilitated by a combination of software-controlled hardware. In certain implementations, at least a portion, and in some cases, all, of a engine can include the processor(s) of one or more computers that execute an operating system, system programs, and application programs, while also implementing the engine using multitasking, multithreading, distributed (e.g., cluster, peer-peer, cloud, etc.) processing where appropriate, or other such techniques. In addition, an engine can itself be composed of more than one sub-engines, each of which can be regarded as an engine, whether collectively or individually.

In the example of FIG. 6, one set of recommended options for scheduling adjustments and recommended changes is depicted. These changes generally include the new course of treatment changes listed in grouping 420. Although not specifically shown in FIG. 6, in certain embodiments a plurality of recommended options, each consisting of their own new course of treatment, can be initially chosen for graphical review. Thereafter, a new recommended course of treatment can be previewed graphically for selection.

In the example of FIG. 6, a recommended option has already been selected and it is therein being described and displayed as including 5 adjustments, namely: “1. Resume Vancomycin @ 5:15”; “2. Move Flush to follow Vancomycin @ 6:45”; “3. Move Gentamacyn to follow Vancomycin Flush @ 13:00”; “4. Move Saline Flush to follow Gentamacyn @ 15:00”; and “5. Adjust Normal Saline to make up the balance to 2.25 mL/hr”. These adjustments can be seen in FIG. 6, with adjustment 1 at 431, adjustment 2 at 432, adjustment 3 at 433, adjustment 4 at 434, and adjustment 5 at 435 respectively. Corresponding previews of adjustments to graphical depictions can be seen as well.

The graphical depiction of the recommended option shown uses a specific color of shading 440 to indicate the removal and addition of infusions or other patient treatments. Further, graphics like arrows 450 are used to assist the user readily distinguish where various infusions or events are being moved. Arrows 450 or similar such graphics are not included in some embodiments. Numerous other more graphically intensive options to alternatively graphically depict these proposed changes are possible and contemplated by this disclosure. For example, proposed changes could be shown with patterns, solid and flashing portions, semi-transparent outlines, side-by side comparison displays, or detailed animations, etc.

Using features as those aforedescribed, for example, it is to be appreciated and understood that an infusion planning system is able to provide clinical decision support for dynamic treatment scheduling. As depicted in FIG. 5, for example, some embodiments will include a GUI 14 and a rescheduling assistance engine 320 that provides a plurality of selectable schedule updates 322 that each include a set of recommended changes to the plurality of patient treatments 304 in accordance with assigned rules governing each of the plurality of patient treatments. Visual previews 330 of the selectable schedule updates 322 are provided to help make any changes easy to understand and readily implement. Certain embodiments may incorporate a drag and drop tool 370 allowing ease of user selection of options corresponding to the selectable schedule updates 322 available. In some embodiments, simply dragging a desired patient treatment modification graphic into the GUI 14 results in a readily reviewable and understandable display.

Accordingly, once the desired modification is reviewed, the user may accept or reject the set of recommended changes 350 by selecting appropriate accept/reject options 360, as shown in FIG. 6. Some embodiments will require approval to certain modifications in scheduling by a physician or other medical professional. Approval may require some type of additional authentication procedure to ensure that the operator of the infusion planning system has the authority to make the requested changes following selection of an Accept option 360.

Certain embodiments will enable modifications in scheduling to automatically occur for minor medical alterations when necessary. Updates can be prompted by the system in some embodiments and recommendations and advice for altering the schedule can be suggested in some embodiments. Certain embodiments may even allow for complete closed loop control over patient scheduling and prescription of treatments within a prescribed set of rules that have been approved by medical professionals. In most embodiments, however, robust and easy to use advising capabilities and easy to use approval procedures will be useful aspects of the disclosed infusion planning system.

Certain embodiments may rely on implementation of the infusion planning system using a computing platform interfaced with a computer network, the computing platform including computing hardware having a processor, data storage, and input/output facilities, and an operating system implemented on the computing hardware. The infusion planning system can also include instructions that, when executed on the computing platform, cause the computing platform to implement a graphical user interface and a rescheduling assistance engine.

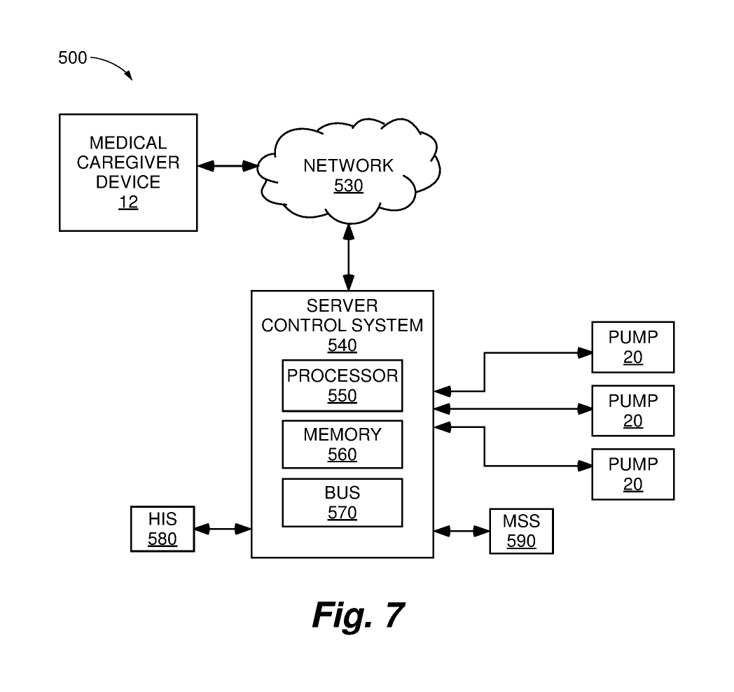

FIG. 7 shows an overview type example of an embodiment of an infusion planning system providing caregivers an easy-to-use system than can be employed to plan, coordinate, and monitor medication deliveries and other medical events. While the GUI 14 of FIGS. 2 and 3 shows one component of the planning system, the system typically utilizes a larger system 500 such as the one depicted in FIG. 7. Numerous other configurations of the hardware and software components of the system are possible. Some possible configurations are depicted in the aforementioned PCT/US2014/044586 patent application, for example.

The medical caregiver devices 12, depicted in FIG. 7 and described throughout this document by example, can comprise a personal computer, PC workstation, laptop, electronic tablet, smart phone, custom controller, server computer, hand held device or other display and processing device providing a GUI 14. The devices 12 can operate with other general purpose computer systems or computer configurations. These medical caregiver devices 12 can be controlled via a keyboard, mouse, or other touch-less or touch-based input devices. Further, inputs can be speech/voice activated or motion activated as well. Personal computers or PC workstations, for example, might be set up at a hospital unit, nurse station, or patient bedside.

For purposes of this disclosure, the terms “Computer,” “Computer system,” “Computing system,” or “Computing platform” can be defined as an electronic device or system of inter-operable electronic devices containing hardware including one or more processors, data storage, input-output devices; and capable of storing and manipulating information according to software instructions carried out by the hardware. It can be one physical machine, or it can be distributed among multiple physical machines, such as by role or function, or by process thread in the case of a cloud computing distributed model. Examples include desktop or mobile personal computers (PCs), smartphones, and tablets, as well as networking devices, such as routers, switches, and the like. Computer systems can be stand-alone devices, or embedded devices that are part of a larger device or system.

Pumps 20 can include a variety of medical infusion pumps. These infusion pumps 20 can include, but are not limited to, peristaltic pumps and syringe pumps, for example. These infusion pumps 20 generally can be used to provide fluids, medication, or nutrition to a patient 16. Infusions made possible can include but are not limited to therapeutic agents; nutrients; drugs; medicaments such as antibiotics, blood clotting agents, and analgesics; and other fluids. The pumps 20 can be used to introduce the medications or fluids into the body of a patient utilizing any of several routes such as, for example, intravenously, subcutaneously, arterially, or epidurally. Infusions can be delivered according to various delivery profiles, such as continuous, intermittent, or patient controlled, for example.

The network 530, utilized by the system can represent a networking environment such as a local area network or wide area network. In a network environment, programming can be stored in the server memory, one or more medical caregiver devices, or other networked component. The network 530 and server enable connection of devices throughout a hospital, medical facility, research environment, laboratory, clinic, administrative offices, or other connection.

The server control system 540 is part of a computing environment and can be considered a general purpose computing device in various embodiments. The server control system 540 can include at least a processor 550, memory 560 and data bus 570, for example. Although the many components are generally shown as residing on a single server or computing device, it should be understood that any number of components can reside on any number of servers or computing devices.

The processor 550 described in the figures and throughout this document can be any programmable device that accepts digital data as input, is configured to process the input according to instructions or algorithms, and provides results as outputs, for example. In an embodiment, processor 550 can be a central processing unit (CPU) configured to carry out the instructions of a computer program. Processor 550 is therefore configured to perform arithmetical, logical, and input/output operations. For purposes of this disclosure, the term “Processor” can be defined as electronic hardware part of a computer system that carries out the instructions of a computer program by performing arithmetical, logical, temporary storage, and input/output operations of the system. Typically, a processor is implemented as a microprocessor (i.e., integrated on a single chip), though this definition includes processor circuits that are implemented on multiple interconnected integrated circuits. Modern-day processors typically include multiple processing cores and can distribute workload among the multiple processing cores.

The memory 560 can comprise volatile or non-volatile memory as required by the coupled processor 550 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the claims.

The data bus 570 manages various parts of the systems described and generally serves as a connection framework for various parts, including the processor and memory. In general, the data bus 570 provides a communications architecture for exchanging information throughout the system. The system data bus 570 can include a memory bus, memory controller, peripheral bus or local bus of various bus architectures. These bus architectures can include, but are not limited, to Industry Standard Architecture (ISA), Extended Industry Standard Architecture (EISA), IBM Micro Channel, VESA Local bus, Peripheral Component Interconnect and others.

The Hospital Information System (HIS) 580 comprises the information or management system of a hospital, with all of its subcomponents and subsystems. The HIS 580 refers to a system providing healthcare related information that is integrated and is accessible by persons at a hospital or healthcare facility to assist in providing patient care. These are often comprehensive, integrated information systems designed to manage the medical, administrative, financial and legal aspects of a hospital and its service processing. The HIS 580 can include or manage electronic medical records for patients. Such electronic records can include up-to-date medical histories, patient data, lab work, test results, prescriptions, imaging and diagnosis information for patients. The HIS 580 can be configured to transmit data to a server for integration into the drug libraries in some embodiments. Likewise, data can be transmitted from a server to the HIS 580 for informational, reporting, or patient care purposes.

The Medication Safety Software (MSS) 590 includes medication information parameters and drug libraries that can be used by medical practitioners, “smart” infusion pumps, and medical equipment to assist in safely controlling the introduction of medicaments to a patient when medical personnel are not continuously present. MSS 590 information can provide information to smart pumps concerning, or imposing, safety limits on medication program parameters such as dose, concentration, and time, etc., for delivery of a particular medication from the pump to a particular patient. Practitioners create and maintain so-called “drug libraries” associated with such safety limits that are utilized by the MSS 590.

Aspects of the present invention can be implemented as part of a computer system. The computer system can be one physical machine, or can be distributed among multiple physical machines, such as by role or function, or by process thread in the case of a cloud computing distributed model. In various embodiments, aspects of the invention can be configured to run in virtual machines that in turn are executed on one or more physical machines. It will be understood by persons of skill in the art that features of the invention may be realized by a variety of different suitable machine implementations.

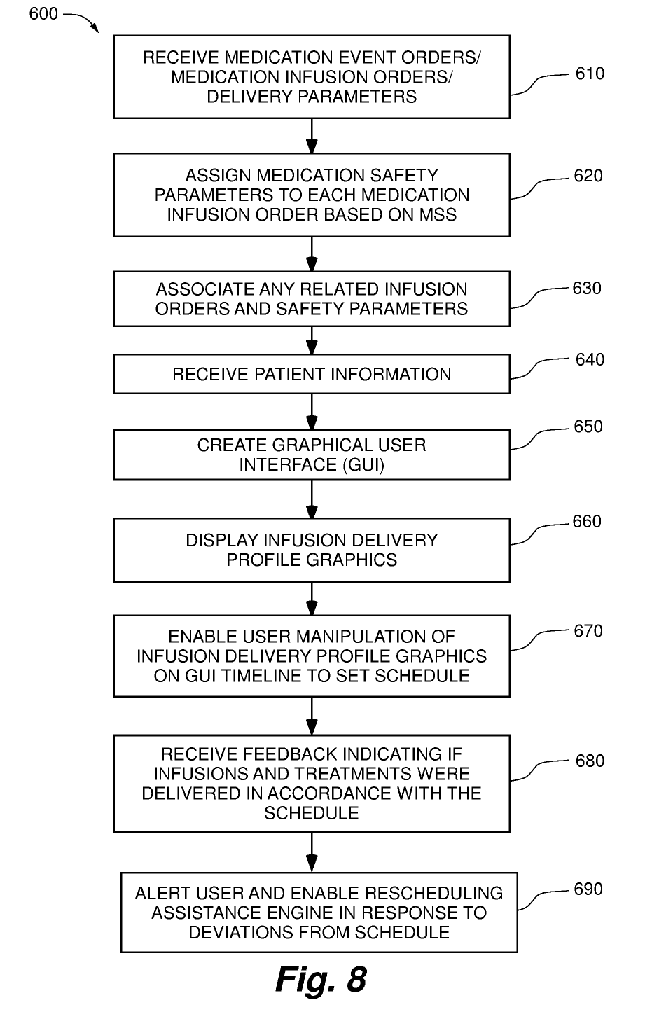

FIG. 8 is a flow diagram of an example of an infusion planning method 600. In general, the system receives medication event orders, medication infusion orders, and other delivery parameters within the system, at 610. At 620, medication safety parameters are assigned to each medication infusion order based on medication safety software programming. At 630, any related infusion orders and safety parameters are associated and at 640 patient information is received by the system. At 650, a graphical user interface is created by the system and at 660 infusion delivery profile graphics (e.g., 110 in FIG. 5) are displayed on the graphical user interface (e.g., 14 in FIG. 5). At 670, the system enables user manipulation of the infusion delivery profile graphics on a timeline 204 shown on the graphical user interface 14 (again, e.g., 14 in FIG. 5) to set a schedule for a patient in a medical care facility. At 680, feedback from the system and patient is received indicating whether infusions and other patient treatments (e.g., 304 in FIG. 5) were delivered consistent with the schedule. At 690, the system alerts the user and enables a rescheduling assistance engine in response to deviations in the schedule.

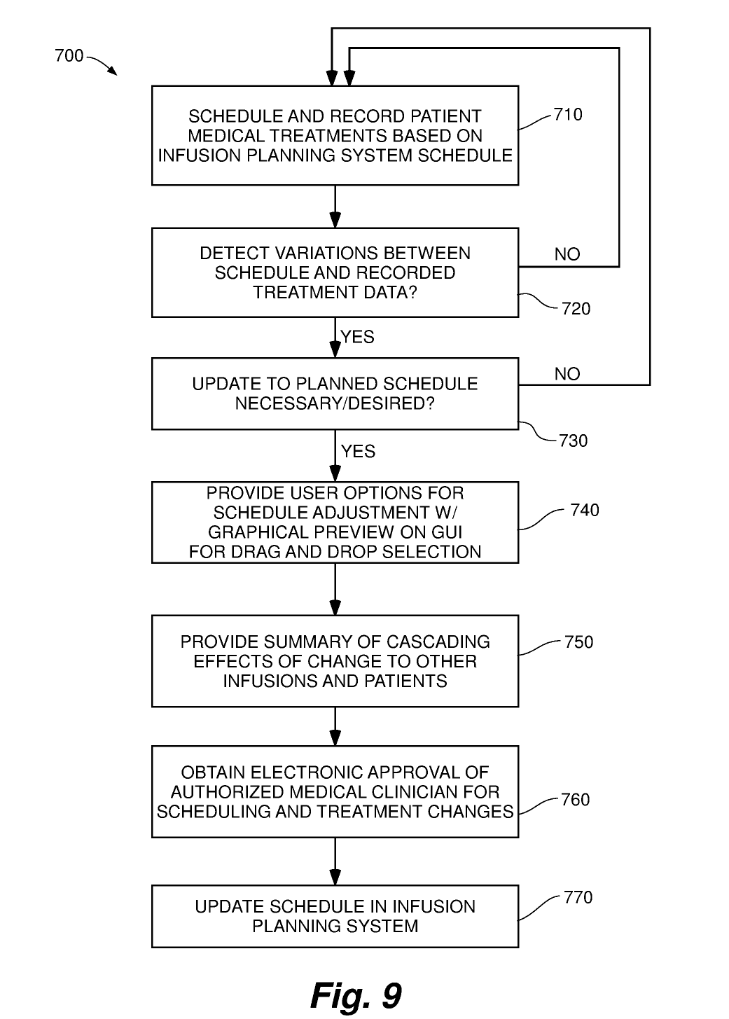

FIG. 9 shows a flow diagram of an example of a method 700 of updating a treatment schedule for a patient in an infusion planning system. Initally at 710, patient medical treatments are scheduled and recorded in an infusion planning system schedule. At 720, the system detects whether there are variations between the record treatment data and the projected scheduled data. If there are variations, the system requests whether to update the future planned schedule of the infusion planning system at 730. If the user desires to make updates, at 740, the system provides the user a set of option for schedule adjustment including a graphical preview on the graphical user interface, in which various options for schedule adjustment can be dragged and dropped into the infusion planning schedule as aforedescribed. At 750, the system provides a summary of cascading effects that the options for schedule adjustment will have on other infusions and patients. At 760, electronic approval of an authorized medical practitioner is sought to approve scheduling and treatment changes. At 770, the system updates the schedule for the infusion planning system.

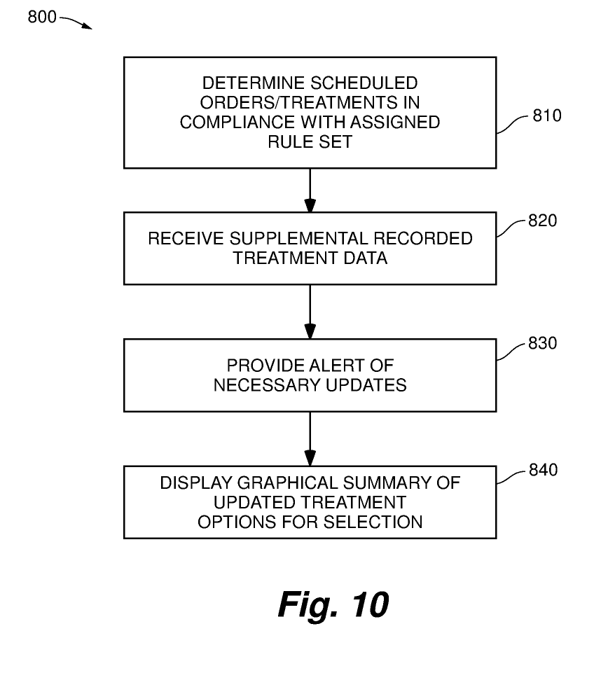

FIG. 10 is a flow diagram of an example of a method 800 of updating schedules in an infusion planning system. First, at 810, the system determines the scheduled treatments and orders to create a GUI displayed schedule in compliance with an assigned set of rules. At 820, supplemental feedback data that has been recorded is received by the system. At 830, alerts are provided where the feedback received indicates that an update is necessary. At 840, a graphical summary of updated treatment options that may be selected by a user is displayed.

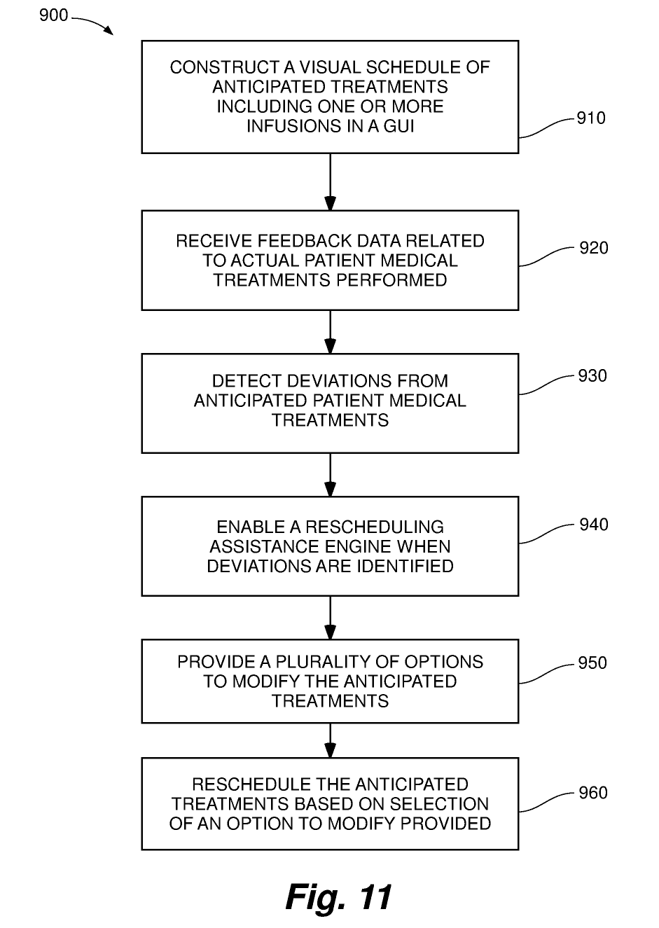

FIG. 11 is a flow diagram of an example of a method 900 of updating a treatment schedule for a patient in an infusion planning system. The method 900 includes constructing a visual schedule of anticipated patient medical treatments including one or more infusions in a graphical user interface of an infusion planning system, at 910. At 920, the method also includes receiving feedback data related to actual patient medical treatments performed on the patient. At 930, the method also includes detecting deviations from the anticipated patient medical treatments based on the feedback data received. At 940, a rescheduling assistance engine is enabled when deviations are identified. At 950, the system provides a plurality of options to modify the anticipated patient medical treatments. At 960, the method also includes rescheduling the anticipated patient medical treatments based on a selection of one of the plurality of options to modify provided by the rescheduling assistance engine.

In some embodiments, at 920 the feedback data can include monitored patient paramenter data in response to the actual medical treatments performed. Further, at 930 the deviations from the anticipated medical treatments can include variations from anticipated monitored patient parameter data. Accordingly, detection of deviations of patient diagnostic parameters from anticipated patient diagnositic parameters can serve to enable the rescheduling assistance engine in certain circumstances.

It is to be appreciated and understood that any embodiments described herein are only examples, and are not intended to limit the scope, applicability, or configuration of the novel and inventive subject matter hereof in any way. Rather, the foregoing detailed description will provide those skilled in the art with an enabling disclosure for implementing one or more embodiments. It should be understood that various changes can be made in the function and arrangement of elements without departing from the scope of the novel and inventive subject matter hereof, as set forth in the appended claims and the legal equivalents thereof.

Embodiments described by example or otherwise contemplated herein are intended to be illustrative and not limiting. Additional embodiments may be within the novel and inventiove subject matter hereof, and the claims. Although examples have been described herein with reference to particular embodiments, workers skilled in the art will recognize that changes may be made in form and detail without departing from the spirit and scope of the novel and inventiove subject matter hereof.

Various modifications may be apparent to one of skill in the art upon reading this disclosure. For example, persons of ordinary skill in the relevant art will recognize that the various features described for the different examples of embodiments can be suitably combined, un-combined, and re-combined with other features, alone, or in different combinations, all within the spirit and scope of the novel and inventive subject matter hereof. Likewise, various features described herein should all be regarded as example embodiments, rather than limitations to the scope or spirit of the novel and inventive subject matter hereof. Therefore, the foregoing written description and accompanying drawings do not limit the scope of the novel and inventive subject matter hereof.

1. An infusion planning system that provides adaptive clinical decision support for dynamic patient treatment scheduling, comprising:
a graphical user interface presenting a time schedule display graphically representing a plurality of patient treatments over time including at least one infusion delivery profile graphic associated with an ordered infusion; and
a rescheduling assistance engine, including:
a user interface display providing a plurality of selectable schedule updates each comprising a set of recommended changes to the patient treatments in compliance with rules governing the patient treatments.

2. The infusion planning system of claim 1, wherein the rescheduling assistance engine provides a selectable graphical visual preview of each of the selectable schedule updates.

3. The infusion planning system of claim 2, wherein the selectable graphical visual preview is shown on the time schedule display.

4. The infusion planning system of claim 2, wherein the selectable graphical visual preview is shown on within the user interface display.

5. The infusion planning system of claim 1, wherein the rescheduling assistance engine requires user authentication to implement one of the plurality of the selectable schedule updates.

6. The infusion planning system of claim 1, wherein one of the plurality of the selectable schedule updates from the rescheduling assistance engine can be implemented in the graphical user interface via a drag and drop tool.

7. The infusion planning system of claim 1, wherein the user interface display of the rescheduling assistance engine prompts a user when system feedback indicating deviations from the plurality of treatments in the time schedule display are recognized.

8. The infusion planning system of claim 1 wherein the user interface display of the rescheduling assistance engine is manually made accessible by a user request.

9. The infusion planning system of claim 1, wherein the rescheduling assistance engine identifies one of the selectable schedule updates as the set of recommended changes that is most preferred.

10. An infusion planning system that provides adaptive clinical decision support for dynamic patient treatment scheduling, comprising:
a computing platform interfaced with a computer network, the computing platform including computing hardware having a processor, data storage, and input/output facilities, and an operating system implemented on the computing hardware; and
instructions that, when executed on the computing platform, cause the computing platform to implement:
a graphical user interface presenting a time schedule display graphically representing a plurality of patient treatments over time;
a rescheduling assistance engine that identifies and graphically presents recommended options for scheduling adjustments on the time schedule display of the graphical user interface that include at least one alternate sequence of revised patient treatments.

11. An infusion planning system that provides adaptive clinical decision support for dynamic patient treatment scheduling, comprising:
a graphical user interface for scheduling a plurality of patient treatments including scheduling a plurality of infusions delivered by at least one infusion pump, the graphical user interface comprising:
a time schedule display comprising a plurality of columns representing time intervals during at least one particular date;
a plurality of infusion bars each representing an ordered infusion for administration;
one or more infusion delivery profile graphics associated with each ordered infusion that depicts both an amount of medication to be delivered to a patient and a length of time period needed for delivery;
wherein the infusion delivery profile graphics are configured for movement within the infusion bar associated with the corresponding ordered infusion, such that the infusion delivery profile graphics are aligned with the columns representing the time intervals over which the ordered infusion is scheduled for delivery.
a rescheduling assistance engine providing a plurality of selectable schedule updates each comprising a set of recommended changes to the plurality of patient treatments in accordance with assigned rules governing each of the plurality of patient treatments;
wherein the graphical user interface provides visual previews of the selectable schedule updates.

12. The infusion planning system of claim 11, wherein the graphical user interface includes a display having historical and real-time parameter feedback of a patient indicative of responses to one or more clinical changes.

13. A rescheduling assistance engine for use within a graphical user interface of a medical scheduling system, including:
a user interface display providing a plurality of selectable schedule updates each comprising a set of recommended changes to one or more patient treatments in compliance with rules governing the one or more patient treatments;
wherein selectable graphical visual previews of each of the plurality of selectable schedule updates are provided;
wherein at least of the one or more patient treatments is an infusion.

14. A method of updating a treatment schedule for a patient in an infusion planning system, comprising:
constructing a visual schedule of anticipated patient medical treatments including one or more infusions in a graphical user interface of an infusion planning system;
receiving feedback data related to actual patient medical treatments performed on the patient;
detecting deviations from the anticipated patient medical treatments based on the feedback data received;
enabling a rescheduling assistance engine when deviations are identified;
providing a plurality of options to modify the anticipated patient medical treatments using the rescheduling assistance engine;
rescheduling the anticipated patient medical treatments based on a selection of one of the plurality of options to modify the anticipated patient medical treatments provided by the rescheduling assistance engine.

15. The method of claim 14, wherein receiving feedback data related to actual patient medical treatments performed on the patient includes receiving responsive monitored patient parameter data, wherein detecting deviations from the anticipated patient medical treatments includes detecting variations from anticipated monitored patient parameter data.

16. An infusion planning system for scheduling medical events including medical infusions, comprising:
a graphical user interface including:
a timeline-based graphical schedule of future patient treatments on a first portion of a displayed timeline; and
a timeline-based graphical schedule of past patient treatments on a second portion of the displayed timeline, configured to display verified past patient treatments and unverified past patient treatments, based on feedback received regarding a set of actual patient treatments administered.

17. The infusion planning system of claim 16, wherein unverified past patient treatments reflect a first appearance distinguishable from the appearance of the verified past patient treatments.

18. The infusion planning system of claim 16, wherein the unverified past patient treatments are shown in grayscale.

19. The infusion planning system of claim 16, wherein an authorized medical clinician is permitted to select and convert unverified past medical events to verified past patient treatments.

20. The infusion planning system of claim 19, wherein the authorized medical clinician is prompted to review unverified past medical treatments at system selected times.

21. The infusion planning system of claim 16, wherein the patient treatments displayed on the graphical user interface contain a user selectable icon which provides a list of settings criteria associated with that particular patient treatment.

22. The infusion planning system of claim 16, wherein the graphical user interface includes a real-time display of patient parameter feedback indicative of the current and past diagnostic states of a patient.

23.-26. (canceled)
